(19) United States
(12) Patent Application Publication
(10) Pub. No.: US 2022/0211267 A1
(43) Pub. Date: Jul. 7, 2022

(54) DEVICE NAVIGATION AND CAPTURE OF MEDIA DATA

(71) Applicant: Spect Inc., Palo Alto, CA (US)

(72) Inventors: Michael Christopher Leung, San Francisco, CA (US); Neil Batlivala, San Francisco, CA (US); Ankur Sudhir Gupta, San Mateo, CA (US); Theodore Leng, Portola Valley, CA (US); Misha Chi, Alameda, CA (US)

(21) Appl. No.: 17/603,579

(22) PCT Filed: Apr. 14, 2020
(86) PCT No.: PCT/US20/28153

§ 371 Date: Oct. 13, 2021

Related U.S. Application Data
(60) Provisional application No. 62/834,626, filed on Apr. 16, 2019.

(51) Int. Cl. A61B 3/14; G06T 7/00; G06T 7/70; G06T 7/593; G06V 10/74

(52) U.S. Cl. CPC A61B 3/14; G06T 7/0012; G06T 2207/30041; G06T 7/593; G06V 10/761; G06T 7/70

(57) ABSTRACT

Methods and systems describe remotely triggering the capture of media data. A navigator device at a server is connected with an operator device over a network, with the operator device being handled by an operator. Streaming media data is received at the server, and concurrently, the incoming streaming media data is analyzed to determine whether a match for one or more predefined visual landmarks can be identified. Upon a determination that a match cannot be identified, instructions are communicated, via the navigator device, for the operator to reposition the operator device. Upon a determination that a match for one or more predefined visual landmarks can be identified, capture of the streaming media data with the visual landmarks is triggered.

### CROSS REFERENCE TO RELATED APPLICATIONS

This application claims priority to U.S. Application No. 62/834,626 filed on Apr. 16, 2019, and entitled “METHOD FOR ACQUIRING OCULAR MEDIA,” which is hereby incorporated by reference in its entirety.

### FIELD OF THE INVENTION

The present invention relates generally to media capture, and more particularly to novel methods and apparatuses for device navigation and capture of media data.

### BACKGROUND

Studies show that within the United States, only 60% of the 30 million known diabetics actually receive their mandatory annual retinal eye screening due to inconvenience, cost, and/or lack of specialist availability. If diabetes-related eye disease is detected late, 40-45% of diabetics may lose their eyesight, despite such diseases being treatable. Early detection and treatment, while the patient is still asymptomatic, can prevent up to 98% of visual loss due to diabetes. Retinal eye exams can also diagnose a plethora of other eye diseases, such as glaucoma, age-related macular degeneration, retinal detachment, and cataracts, as well as whole body problems.

Diabetics are recommended to visit an ophthalmologist on a yearly basis to get their retina examined. However, very few of them actually get the procedure done, since there are a limited number of ophthalmologists and retinal specialists. The ability to screen for retinal diseases at the primary care site (e.g., emergency room, primary care doctor, corporate wellness site, pharmacy, and eventually at home) would allow for large cost savings, by alleviating dependence on specialists for basic vision diagnostics. There are magnitudes more nurses, medical assistants and primary care doctors than there are specialists. Retinal examinations can be separated into two steps: a data acquisition step, and a data interpretation step. Today, both steps are typically performed in the optometry or ophthalmology office. The data interpretation step (e.g., medical grading, or annotation) can be performed by, e.g., ophthalmologists or retinal specialists with or without the aid of machine learning or other artificial intelligence methods. It can also be performed by a machine without the input of a person. The interpretation portion, such as grading or reading of an image, can be done asynchronously; that is, potentially hours or days after the initial data acquisition step; or in the case of machines, in real time or substantially real time, for example, completed in a few minutes after the acquisition.

Current solutions typically involve the use of special “fundus cameras”, which are large, expensive, require specialized training, and are generally limited to specialists' offices, such as ophthalmologists.

Therefore, a need exists in the field for a device which is inexpensive and simple to use outside of the specialists' offices. The source of the problem, as discovered by the inventors, is a lack of small, inexpensive devices for retinal examination which do not require specialized training to operate, and which can allow for the data acquisition step of retinal examinations to be achieved in a primary care office or even at home.

### SUMMARY OF THE INVENTION

Herein, we describe systems and methods for triggering the capture of media data (e.g., images, videos, or any other suitable media).

In some embodiments, the media data is ocular image data relating to a patient's eye, such as images of the retina. In some embodiments, the image data is captured with the use of a mobile device. The mobile device is operated by a local user (the “operator”) with guidance from an expert (the “navigator”) who is able to view the data capture in real time or substantially real time. In some embodiments, the image data is streamed to the navigator device as a series of incoming images or video as the navigator views the data. In some embodiments, the operator is guided by the use of auditory instructions and visual cues. In some embodiments, the expert initiates the capture of the media, whereas in other cases, the local operator initiates the capture. In some embodiments, the device automatically initiates the capture, based on, e.g., an algorithm, artificial intelligence, or other suitable method. In some embodiments, some or all of the navigator's and/or operator's tasks can be automatically or semi-automatically performed, in whole or in part, by the navigator device, operator device, server, or any combination thereof. In some embodiments, once the media has been captured, it is transmitted and stored in cloud services where it can then be retrieved for interpretation at a later point in time.

In one embodiment, the system connects, over a communications network, a navigator device with an operator device. One or both devices may be located on or accessed via a server accessible via the network. In some embodiments, a base device or dock is configured to serve as an intermediary or connecting device between the server and one or both of the devices (e.g., the operator device or the navigator device). In some embodiments, remote data transfer between the devices is established. In some embodiments, additionally or alternatively to remote data transfer, data can be cached, queued or stored for later upload (e.g., at a specified time of day, or upon connection to the server via a more stable or strong connection, such as a wired LAN connection). In some embodiments, data is stored locally on one or both devices and transfer is performed manually, via a wired transfer, or via some other method other than remote data transfer.

In some embodiments, the operator device is handled by an operator, and the operator device includes a speaker and microphone configured to facilitate two-way communications with the navigator device over the network. The system receives, at the server, streaming image data from the operator device. Concurrent to receiving the streaming image data, the system continuously analyzes the streaming image data to determine whether a match for one or more predefined visual landmarks can be identified within the streaming image data. If the landmarks cannot be identified, the system communicates, via the navigator device and/or navigator, instructions for the operator to reposition the operator device. If the landmarks can be identified, the system triggers the streaming image data with the landmarks to be captured.

In some embodiments, concurrent to receiving the streaming image data, the system analyzes the streaming image data to determine whether a predefined threshold for image quality is met or exceeded. In some embodiments, if the threshold is neither met nor exceeded, the navigator device and/or navigator sends one or more instructions to the operator device for the operator to reestablish connection to the media stream or resend the streaming media at a higher quality.

The features and components of these embodiments will be described in further detail in the description which follows. Additional features and advantages will also be set forth in the description which follows, and in part will be implicit from the description, or may be learned by the practice of the embodiments.

### BRIEF DESCRIPTION OF THE DRAWINGS

FIG. 1A is a diagram illustrating an exemplary environment in which some embodiments may operate.

FIG. 1B is a diagram illustrating an exemplary computer system that may execute instructions to perform some of the methods herein.

FIG. 2A is a flow chart illustrating an exemplary method that may be performed in some embodiments.

FIG. 2B is a flow chart illustrating additional steps that may be performed in accordance with some embodiments.

FIG. 3A is a flow chart illustrating an exemplary method of connecting an operator device to a navigation server, in accordance with some embodiments.

FIG. 3B is a flow chart illustrating an exemplary method of interpreting image data after an examination, in accordance with some embodiments.

FIG. 4 is a diagram illustrating one example embodiment of an implementation on the operating device in accordance with some of the systems and methods herein.

FIG. 5A is a diagram illustrating one example embodiment of a time-based flow of the navigation process, in accordance with some of the systems and methods herein.

FIG. 5B is a diagram illustrating one example embodiment of a time-based flow of the navigation process, in accordance with some of the systems and methods herein.

FIG. 5C is a diagram illustrating one example embodiment of a time-based flow of the navigation process, in accordance with some of the systems and methods herein.

FIG. 5D is a diagram illustrating one example embodiment of a time-based flow of the navigation process, in accordance with some of the systems and methods herein.

FIG. 5E is a diagram illustrating one example embodiment of a time-based flow of the navigation process, in accordance with some of the systems and methods herein.

FIG. 5F is a diagram illustrating one example embodiment of a time-based flow of the navigation process, in accordance with some of the systems and methods herein.

FIG. 5G is a diagram illustrating one example embodiment of a time-based flow of the navigation process, in accordance with some of the systems and methods herein.

FIG. 6 is a diagram illustrating an exemplary computer that may perform processing in some embodiments.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented in whole or in part by a computer system. A computer system may include a processor, a memory, and a non-transitory computer-readable medium. The memory and non-transitory medium may store instructions for performing methods and steps described herein.

As used herein, “ocular” can refer to the retina, the fundus, optic disc, macula, iris, pupil, lens, vessels, vitreous or other eye-related anatomical components. “Media”, or “image data” as used herein, can refer to one or a combination of photo or photos, video or videos, audio, audiovisual, sequence of photos, or other digital information or digital media in general. “Patient” refers to a person being examined, including, in some embodiments, a person whose ocular features are being examined. “User” or “operator” refers to a person who is handling, operating, manipulating, directing, or otherwise making direct physical use of the device. In some embodiments, the user or operator is the person who is holding and orienting the device relative to the patient. In some embodiments, the operator may also be the patient. “Navigator” refers to someone or something that may be guiding or aiding the operator in the procedure. “Specialist” refers to someone or something that may be able to read and diagnose ocular media, such as an ophthalmologist, retinal specialist, or algorithm. A specialist can also be an someone or something that is able to read, interpret, evaluate, grade, assess information that is presented to them, either in person, or in Media; such as an engineer, architect, building inspector.

The “operator device” as used herein refers to a device or multiple connected devices configured to capture or receive media data as well as to transmit media data. In some embodiments, the operator device includes one or more communication enabled components and one or more media capture components (such as a camera). The communication enabled component(s) allow for communication over a network with one or more local or remote devices or servers. In some embodiments, the operator device is, or includes components of, a smartphone that has access to a camera, a speaker, and a microphone, and is capable of transmitting information. The operator device alternatively is, or includes components of, a laptop, desktop, tablet, wearable device, virtual reality and/or augmented reality device, camera, or any other suitable device. In some embodiments, the optical hardware component(s) include one or more optical lenses configured in such a manner as to enable a camera on or connected to the device (e.g., a built-in camera of a smartphone) to focus and capture images and videos. In some embodiments, the optical hardware component(s) include an opthalmoscope or retinal camera. In some embodiments, the communication enabled component(s) and optical hardware component(s) may be components of the same device, while in other embodiments they may be components of separate devices which are physically coupled or configured to communicate with each other locally or remotely.

The “navigator device” as used herein refers to any communication-enabled device by which a navigator, i.e., a person serving as an agent or expert assisting the operator in performing the examination, or an automated system within the navigator device, or a combination of a navigator and semi-automated system, may perform some or all aspects of the navigation server in helping the operator in directing the operator device with respect to the patient and in triggering capture of relevant image data. The navigator device may be, e.g., a mobile phone, laptop, desktop, tablet, wearable device, virtual reality and/or augmented reality device, or any other suitable communication-enabled device.

### I. Exemplary Environments

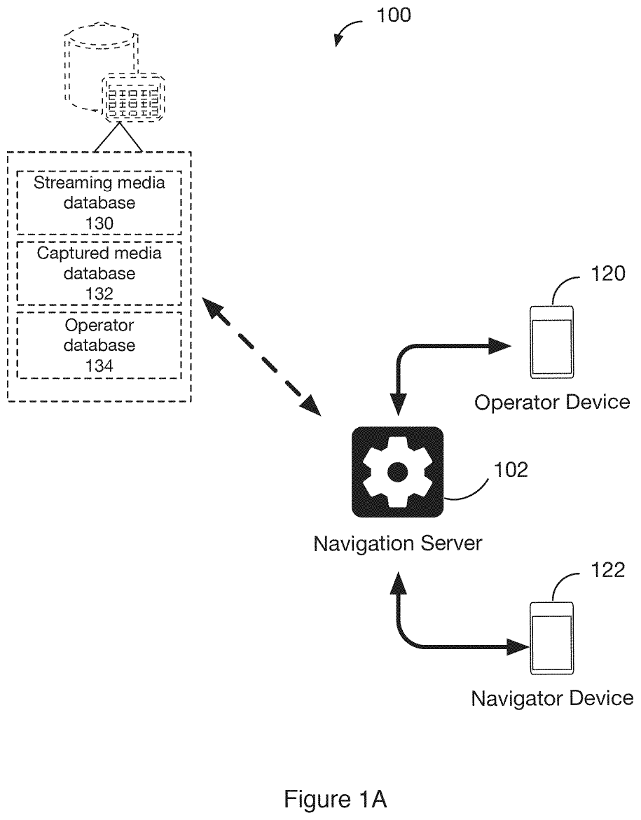

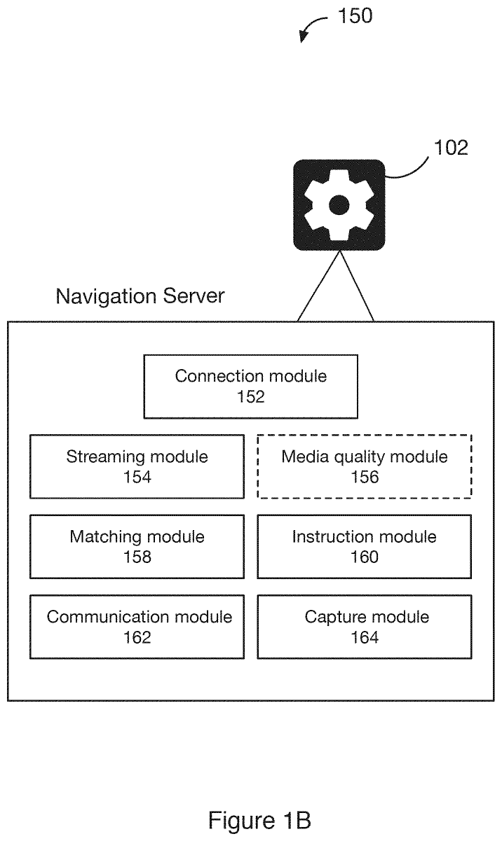

FIG. 1A is a diagram illustrating an exemplary environment in which some embodiments may operate. In the exemplary environment 100, an operator device 120 and a navigator device 122 are connected to a navigation server 102. The navigation server 102 is optionally connected to one or more optional database(s), including a streaming image database 130, captured image database 132, and/or operator database 134. One or more of the databases may be combined or split into multiple databases. The operator device and navigator device in this environment may be smartphones, computers, tablets, or any other suitable device.

The exemplary environment 100 is illustrated with only one operator device, navigator device, and navigation server for simplicity, though in practice there may be more or fewer operator devices, navigator devices, and/or navigation servers. In some embodiments, the operator device and navigator device may communicate with each other as well as the navigation server. In some embodiments, one or more of the operator device, navigator device, and navigation server may be part of the same computer or device. In some embodiments, the operator device includes two or more devices, including a communication-enabled device and an optical hardware device, as described above.

In an embodiment, the navigation server 102 may perform the method 200 or other method herein and, as a result, provide navigation and capturing of media or image data. In some embodiments, this may be accomplished via communication with the operator device, navigator device, and/or other device(s) over a network. In some embodiments, an application server or some other network server may be included. In some embodiments, the navigation server 102 is an application hosted on a smartphone, computer, or similar device, or is itself a smartphone, computer, or similar device configured to host an application to perform some of the methods and embodiments herein.

Operator device 120 is a device for capturing and sending media or image data to the navigation server and/or navigator device. In some embodiments, the operator device 110 enables the operator to perform some or all of a retinal examination. The operator device 110 is described in further detail above and throughout the specification.

Navigator device 122 is a device for assisting the operator and/or operator device in capturing relevant media or image data by aiding the operator with navigation instructions or other instructions as needed. The navigator device 122 is described in further detail above and throughout the specification.

Optional database(s) including one or more of a streaming image database 130, captured image database 132, and/or operator database 134 function to store and/or maintain, respectively, streaming image data, captured images, and operator information, including operator account or device information. The optional database(s) may also store and/or maintain any other suitable information for the navigation server 102 to perform elements of the methods and systems herein. In some embodiments, the optional database(s) can be queried by one or more components of system 100 (e.g., by the navigation server 102), and specific stored data in the database(s) can be retrieved.

FIG. 1B is a diagram illustrating an exemplary computer system 150 with software modules that may execute some or all of the functionality of the navigation server 102 as described herein.

Connection module 152 functions to connect the operator device to the navigation server, the navigator device to the navigation server, and/or the operator device to the navigator device.

Streaming module 154 functions to receive streaming image data from the operator device to be received by the navigation server and/or navigator device, and/or send streaming image data from the operator device.

Optional image quality module 156 functions to adjust image quality within the operator device, navigator device, and/or navigation server. In various embodiments, the image quality module may convert low resolution images into high resolution images, convert high resolution images into low resolution images, or a combination of the two on various devices.

Matching module 158 functions to determine whether a match can be identified within the streaming image data for preidentified visual landmarks within the received image data, and performs steps in response to the determination, as described in further detail below.

Instruction module 160 functions to send instructions from the navigator device to the operator device in order to assist the operator in one or more tasks, including providing navigation or direction to guide the operator in use of the operator device.

Communication module 162 functions to enable various aspects of communication between the operator device and the navigator device, including, in some embodiments, vocal communication between the devices.

Capture model 164 functions to enable local or remote trigger of capture of streaming image data on the operator device, or capture of streaming image data on the navigator device and/or navigation server.

The above modules and their functions will be described in further detail in relation to an exemplary method below.

### II. Exemplary Method

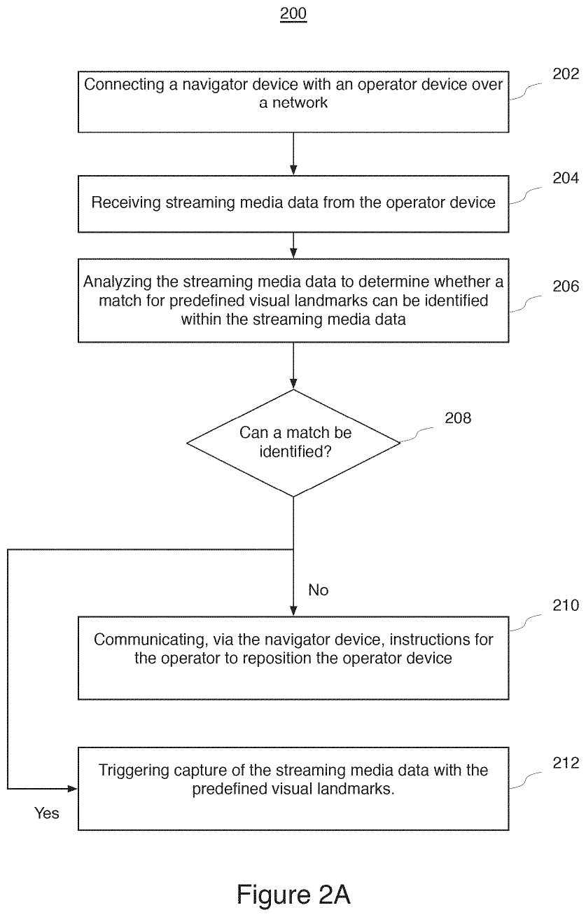

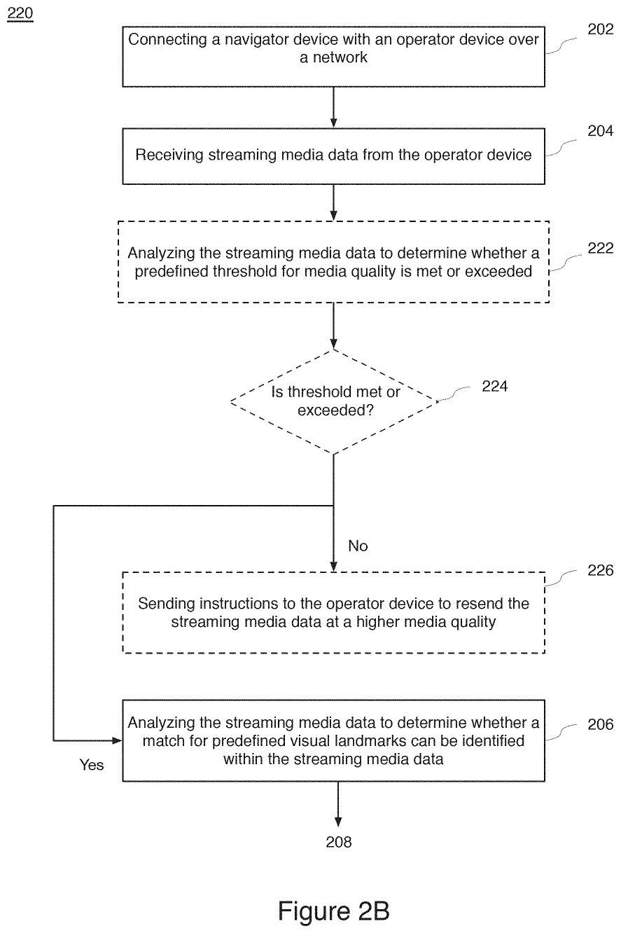

FIG. 2A is a flow chart illustrating an exemplary method that may be performed in some embodiments.

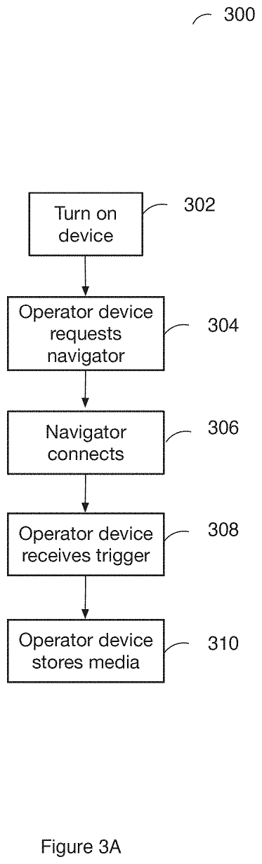

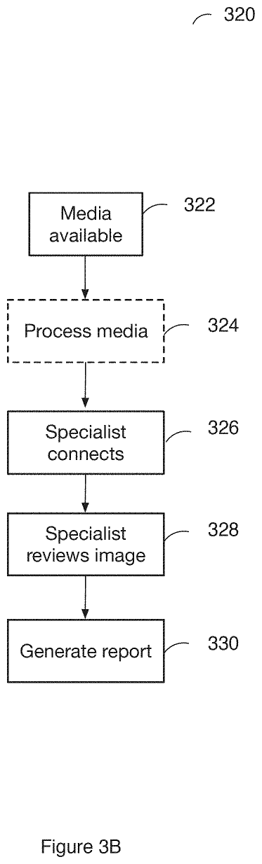

At step 202, the system connects, over a communications network, a navigator device with an operator device. One or both devices may be located on or accessed via a server accessible via the network. The process by which a navigator device connects to an operator device is described in further detail with respect to FIG. 3A below.

At step 204, the system receives streaming image data from the operator device. In some embodiments, the operator device captures image or video data, or other media data, with a camera functionality. In some embodiments, the operator device receives information input from some external source. In some embodiments, the operator device continuously, constantly receives new information input as it streams in. In some embodiments, this information input takes the form of media blocks of information. As information input is received by the operator device, the operator device concurrently sends streaming image data to the navigator device. In some embodiments, this streaming image data is received by the navigator device in real time or substantially real time. In some embodiments, the operator device uses video and/or image compression techniques to reduce the image quality of the information input before it is uploaded to the navigation server and/or the navigator device. The compression may also happen on the navigation server. This allows the images to be received in streaming form, in real time or substantially real time.

At step 206, the system continuously analyzes the streaming image data to determine whether a match for predefined visual landmarks can be identified within the streaming image data. In some embodiments, the visual landmarks relate to the examination procedure and to, e.g., the visual features or parts of anatomy which the operator seeks to capture within the final captured image(s). In some embodiments, the analysis of the streaming image data is performed using deep learning algorithms or networks, machine learning models, or some other form of artificial intelligence.

At decision point 208, the system determines whether a match can be identified. This determination can be performed by, e.g., a human, algorithm, artificial intelligence model, any other suitable human or non-human method, or any combination thereof.

At step 210, the system determines that a match cannot be identified, and communicates, via the navigator device, instructions for the operator to reposition the operator device. In some embodiments, the navigator device communicates these instructions via the navigator sending voice commands (e.g., through a microphone connected to the navigator device) representing instructions for navigating and guiding the operator device to capture relevant image data relating to the predefined visual landmarks. In some embodiments, the navigator may send visual information (e.g., arrows, diagrams, etc.), text information, vibrations or other tactile/haptics, light flashes, or any other suitable form of information for navigation and guidance. The streaming image data continues to be received and viewed by the navigator as the operator guides the operator device according to the instructions. In some embodiments, the instructions are generated and sent automatically or semi-automatically by an automated system. In some embodiments, the operator can send voice statements (e.g., vocalized questions) or other statements to the navigator and/or navigator device, via a microphone on the operator device or other method. For example, the operator can ask for clarification from the navigator, ask questions, point out an obstacle or difficulty in performing one or more tasks, or any other suitable voice statements. In response, the navigator and/or navigator device can provide instructions based on the statements to further guide or assist the operator.

At step 212, the system determines that a match can be identified, and triggers capture of the streaming image data with the predefined visual landmarks. In some embodiments, an automated system automatically detects that the predefined visual landmarks are present in one or more of the streamed image data, and triggers capture. In some embodiments, triggering capture involves sending a signal or notification to the operator device that one or more media blocks are to be captured and stored in the device's storage as well as uploaded to the navigation server. In some embodiments, high quality images, which are higher quality than the low quality streamed image data received by the navigator device, are stored in the device memory as well as uploaded to the navigation server.

FIG. 2B is a flow chart illustrating additional steps that may be performed in accordance with some embodiments. Steps 202, 204, and 206 are as described in FIG. 2A. Concurrent to receiving the streaming image data from the operator at step 204, in some embodiments, additional steps may be performed.

At optional step 222, in some embodiments, the system analyzes the streaming image data to determine whether a predefined threshold for image quality is met or exceeded. This analysis is performed in addition to the analysis to determine a match for predefined visual landmarks. In some embodiments, the predefined threshold is set in accordance with an image quality necessary to identify the visual landmarks to a certain predefined degree of confidence. In some embodiments, the predefined threshold is set by a deep learning network, machine learning model, or other form of artificial intelligence.

At optional decision point 224, the system determines if the threshold is met or exceeded.

At optional step 226, in some embodiments, if the system determines that the threshold is neither met nor exceeded, the system sends instructions to the operator device to automatically process streaming image data on the operator device to improve image quality. In some embodiments, the navigation server or navigator device can send instructions to the operator device to automatically process the streaming image data via instructions to an application on the operator device. Alternatively, in some embodiments, the navigation server automatically processes the streaming image data to improve the image quality. In other embodiments, the navigator will instruct the operator to retry and capture the image data again.

In some embodiments, if the system determines that the threshold is met or exceeded, the system proceeds to step 206 of FIG. 2A.

### I. The Examination

FIG. 3A is a flow chart illustrating an exemplary method of connecting an operator device to a navigation server, in accordance with some embodiments.

When a patient wishes to get their retina examined, they can find a user (“operator”) in possession of the operator device who is available to perform the procedure. In some embodiments, the operator may be the patient themselves. In differing embodiments, the patient's eye(s) may or may not be dilated. In one embodiment where the patient's eyes are not dilated, the communication-enabled device may use light in the far red and infrared region. The camera sensor of the communication-enabled device is able to see the far red/infrared light, but the light would be in a wavelength which is not visible by the unaided human eye.

At step 302, the operator may turn on (activate) the operator device. In some embodiments, the operator is automatically identified and/or authenticated on the operator device. In some embodiments, the operator must sign on to the device, pass a verification process, or otherwise be authenticated within the operator device before continuing.

At step 304, the operator device then requests the navigator. In some embodiments, the operator device generates a new patient examination session. As part of the patient examination session, a new communication “room” is generated. In varying embodiments, the communication room can be established or maintained by an existing third party communication protocol, telecommunications software, remove video or conferencing service, or a communication protocol specific to the navigation system. The communication room is associated with the patient examination session. In some embodiments, a new room is generated automatically upon patient information and/or examination information are entered or registered into the system. The information being entered triggers the room generation process. In some embodiments, the room enables transmission or visual display of media data, such as streaming images or video. For example, in some embodiments, the operator device captures and transmits audio and video, the navigator device connects to the room and one or more media streams, and the navigator device transmits audio from the navigator, receives video from a video stream, and otherwise communicates and receives communication with respect to the operator device. In some embodiments, the creation of the room happens on server well in advance of any party needing to connect, e.g., a minute before either the navigator device or the operator device attempts to establish a connection, or in a different embodiment, days or weeks beforehand, when the appointment is initially scheduled.

At step 306, the operator device is connected (in varying embodiments, automatically or with the aid of the operator) with a navigation device or other support system over a wireless communications network. In some embodiments, the navigation device is associated with a navigator (i.e., expert), while in other embodiments, the navigation system performs the navigator's tasks in an automated fashion. The operator device, navigator device, and/or a server open a two-way communication pathway between the operator and the navigator. In varying embodiments, the operator device can open this communication pathway via Wi-Fi, cellular network, or other internet methods. The navigator will see the video captured by the operator device, which is received as streaming image data and displayed on a screen of the navigator device. In varying embodiments, the navigator and/or navigation device may receive additional information, including but not limited to: audio information, sensor data located within the communication device, such as gyroscopic and accelerometer data, location information (such as GPS), and/or sensor data located within the operator device, such as battery charge status, device operation mode, position and proximity sensors, light sensors and power meters, or any other suitable information.

In varying embodiments, the operator will receive information from the navigator, which may include auditory information, visual information, and/or haptic information. Auditory information can include the navigator verbally giving the operator instructions via voice communications. Visual information can include displaying images on the screen, such as arrows indicating directions to move the operator device in, displaying graphs to guide the operator on the distance or location, and/or other visual cues to give guidance and/or feedback to the operator and patient. In some embodiments, visual cues may include light flashes or variance in the light intensity or frequency of an indicator light. Auditory cues can include voice feedback from the navigator, electronic generated noises (e.g., “beeps”) similar to sonar or echolocation, wherein varying the intensity or frequency could indicate that the device is close or far to the desired location, as well as how close or how far. Haptic information can include vibrations that the operator can feel while holding the operator device, without needing to look at or further interact with the operator device.

In some embodiments, the navigator may guide the operator through the following series of steps. If the operator is familiar with the procedure, the operator may not need the guidance of the navigator.
a. The patient is asked to look away, while the operator positions the operator device at the proper distance from the patient's eye and face.
b. The operator guides the operator device toward the correct working distance. Correct working distance may be achieved by minimizing a light spot on the patient to a single point on the patient's sclera. The optical configuration of the operator device should cause the light spot to focus down to a single point before diverging again, as the axial distance is varied. This is done because light exposure onto the sclera causes minimal discomfort to the patient.
c. When the working distance is approximately correct, the patient is asked to look forward.
d. The light focus spot should now be located on the patient's lens or cornea, with the light illuminating inside the pupil.
e. The navigator may guide the operator into position, by giving the operator information on how to optimally position the operator device to capture the desired media.
f. The navigator may trigger the operator device to capture the desired media. In one configuration, the media captured are still images. In some embodiments, the media captured are motion pictures (video). In some embodiments, the media captured are a consecutive or non-consecutive sequence of still images (e.g., frames of a video).
g. The operator may get some feedback from the navigator to indicate a successful examination has occurred.
h. The media is stored in a location that can be accessed at a later point. In some embodiments, the location can be, e.g., the navigation server 102, streaming media database 130, or captured media database 132.

For step e, the navigator is able to guide the operator based on the information that the navigator sees in real-time. In varying embodiments, information that is provided to the navigator to assist in the guiding can include the video or image stream captured from the operator device, multi-dimensional data (e.g., gyroscopic data, magnetometer data and/or accelerometer data) describing the orientation, direction, movement and/or acceleration of the operator device, and/or distance measurements from a proximity sensor to understand the distance between the operator device and the patient.

In some embodiments, the communication-enabled device has a distance (proximity) sensor which may be an ultrasonic distance sensor, a time-of-flight (optical) sensor or the like, where the distance between the operator device and the patient is determined. The distance sensor can also be optical-based, laser interference-based, LIDAR-based, or based on any other suitable distance measurement technique. In some embodiments, the distance measurements can be used by the navigator, navigator device, and/or navigation server to guide and assist the operator.

In some embodiments, the navigator is substituted by an automated navigator system, via one or more computer algorithms. The automated navigator system is configured to, based on sensor data, previous instructions, and/or knowledge of the current situation, be able to guide the operator into the correct position. In varying embodiments, the automated navigator system may be operating remotely on the navigator device or navigation server, or may be operating on the operator device. In some embodiments, the algorithm may be a deep learning neural network, machine learning model, or any other suitable form of artificial intelligence.

In some embodiments, the operator triggers the capture and acquisition of the media, through a button on the device, a foot pedal or other inputs. In other embodiments, the navigator issues the trigger. The navigator, using the two-way communication pathway, may be able to get an understanding of the situation, and can send a trigger to the operator device when the navigator believes good conditions exist that may result in the capture of favorable media.

To facilitate rapid data transfer, the media quality (resolution) that is streamed to the navigator can be of lower quality than the one received and stored by the operator device. When the operator device receives the trigger event, the operator device may store and upload a higher resolution version of the captured media.

For example, low resolution can mean video streaming resolutions, such as 180p, 280p. High resolution can mean 1080p, 4K, etc. As internet capabilities advance, 1080p can mean low resolution, as 8K and 16K will become society's understanding of high resolution.

In some embodiments, for step f, if the media captured is a video, the trigger event can be a start recording, and a second trigger event can stop the recording. In another embodiment, the trigger event can start the capture of the next few seconds of video (e.g. 5 seconds). In another embodiment, the trigger event can capture the previous few seconds before the trigger event.

In this embodiment, the navigator may be able to see a low quality or high-quality video stream from the operator device. The last few seconds of high-quality media stream can be buffered into the device RAM, or other types of volatile memory, located on or proximal to the operator device, and can be erased or overwritten when it expires. Expiration can be because media is older than a predefined amount of time, for example older than 1 minute. Expiration can be because the operator device has run out of memory space, and newer media is available. When the operator device receives the capture trigger, the operator device can store the past and or future media stream.

In some embodiments, the operator device can store the media by saving to the non-volatile memory, or can transmit the media to the cloud or other storage location.

In some embodiments, after receiving the capture trigger, the 3 seconds of video before the capture trigger, as well as 2 seconds after the trigger is preserved and transmitted to the server. In another embodiment, the last 5 seconds of video are preserved.

### II. After Examination

FIG. 3B is a flow chart illustrating an exemplary method of interpreting image data after an examination, in accordance with some embodiments.

At step 322, after the examination has taken place, the media is available and can be used for interpretation and grading. In some embodiments, the media is stored in an accessible location, such as a cloud server. The specialist is able to access the media to review and interpret at some time in the future. Interpretation includes reading the image and identifying the presence or absence of certain eye conditions, such as, e.g., hemorrhages, cotton-wool spots, retinal detachment, or any other suitable eye conditions which can be identified based on reading the image. Interpretation may also include the diagnosis of several eye diseases based on the observations, such as, e.g., diabetic retinopathy, glaucoma, age-related macular degeneration (AMD), or any other suitable eye disease which can be diagnosed based on the observations.

At optional step 324, in some embodiments, if the media is a video or sequence of photos, it could be processed automatically or manually, and preferred images can be extracted from the video. In varying embodiments, the images can be selected by a person or by an automated system. In some embodiments, the images may be selected based on a predetermined set of parameters, such as, e.g., sharpness, color, blur, presence or absence of reflections or other artifacts. In another embodiment, the images are automatically selected by a computer vision algorithm.

In some embodiments, the media may also be processed automatically or manually in order to enhance the image. Such enhancements may include, e.g., adjusting the color balance, adjusting the brightness, adjusting the contrast. These enhancements can also be spatial, such as to correct, e.g., spherical aberrations, chromatic aberrations, astigmatism, pinhole and fisheye distortions, or any other suitable spatial enhancements.

At step 326, a specialist interacts with the server in order to perform interpretation and/or grading of the image or images. In some embodiments, the specialist can be an ophthalmologist or other medical professional. In other embodiments, the specialist can be a machine learning algorithm or other automated system.

In other embodiments, the human specialist can be augmented or assisted by a machine learning algorithm. In some embodiments, the machine learning algorithm is configured to overlay a heatmap over the media, guiding the specialist to look at certain features present on the media.

At step 328, the specialist reviews the image in order to interpret or grade it. In some embodiments, the specialist reviews additional sensor and data output in addition to the media. For example, the specialist could review the video in combination with the operator device orientation, obtained from multi-dimensional data (e.g., gyroscope and accelerometer data). This may enable the specialist to, e.g., understand multi-dimensional structures seen in the video. In some embodiments, the system may automatically generate multi-dimensional surface topology maps, stereoscopic structures, or any other multi-dimensional materials as a result of receiving the multi-dimensional data.

At step 330, the specialist or automated system can then generate a report with the results, and can share the results with the operator, the medical clinic, and/or the patient.

### III. Device Implementation

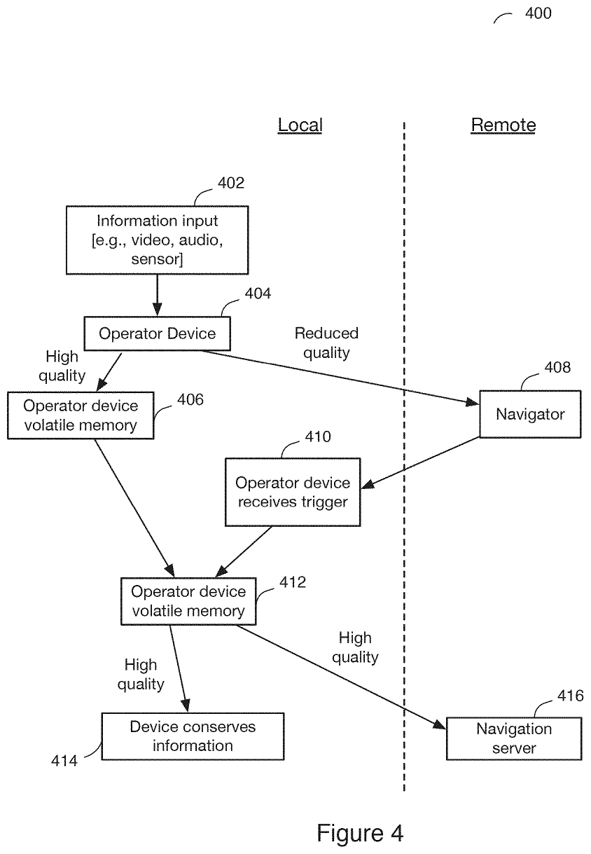

FIG. 4 is a diagram illustrating one example embodiment of an implementation of the navigation process on the operating device in accordance with some of the systems and methods herein.

During the navigation process, the operator device 404 constantly receives information input 402 involving media (e.g., video, audio, sensor, or other media input). The operator device buffers the high quality (e.g., full resolution) images on operator device volatile memory 407, which takes the form of device random access memory (RAM) or other temporary memory. The operator device also sends video in the form of streaming image data to the navigator device 408, in real time or substantially real time concurrent to the storage of the high quality images. This streaming image data is of reduced quality. In some embodiments, video and/or image compression techniques are employed to reduce the quality and ensure the streaming can occur in real time or substantially real time. The last few seconds of high-quality media stream are buffered into the device RAM or other types of volatile memory, located on the device, and can be erased or overwritten when it expires. Expiration can occur because media is older than a predefined amount of time, for example, older than one minute. Expiration can also occur because the device has run out of memory space, and new media is available.

Using the reduced quality video feed, the navigator 408 is able to guide the user on operation, including, e.g., instruction the user to move the operator device in a particular direction. The navigator is also looking for a good capture of the area of interest. When the navigator sees the area of interest, the navigator sends a trigger to the device.

When the device receives the trigger event at 410, the device may store and upload a higher resolution version of the captured media. The higher resolution version of the media is available in the operator device volatile memory at 412, and the device conserves the information at 414. The higher resolution version is also uploaded to the navigation server 416. In some embodiments, the navigator can verify and validate that the media received is of good quality upon uploading.

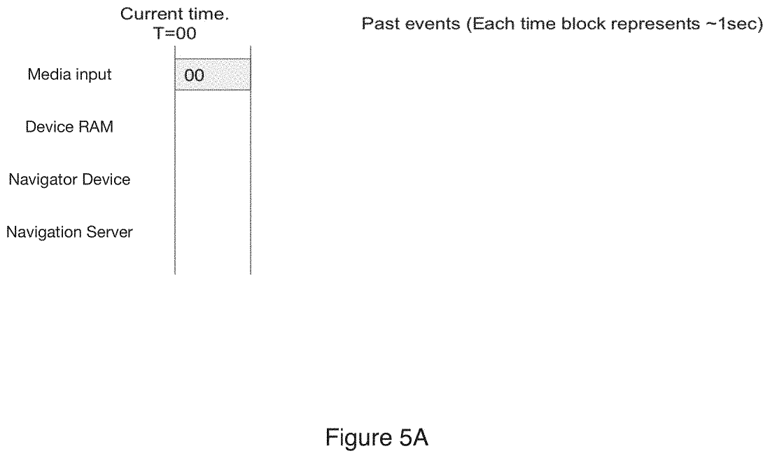

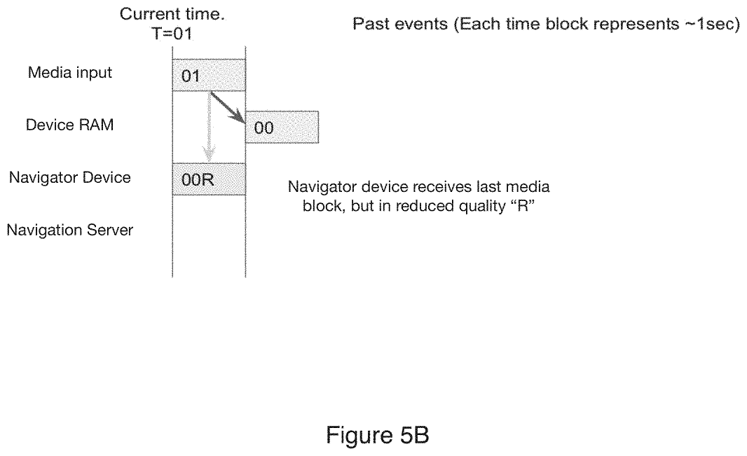

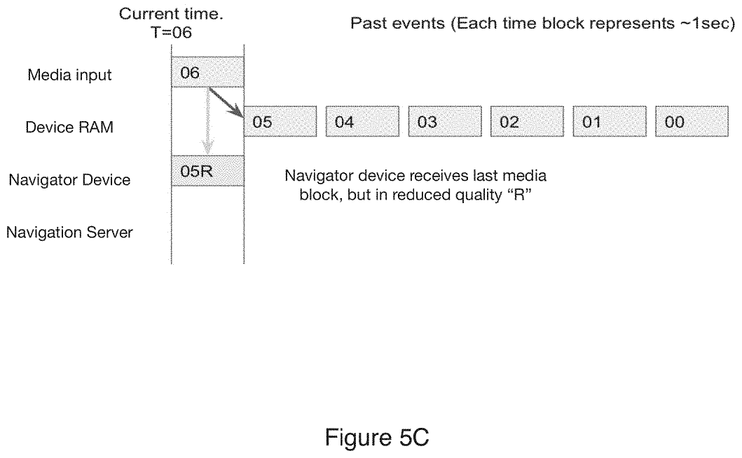

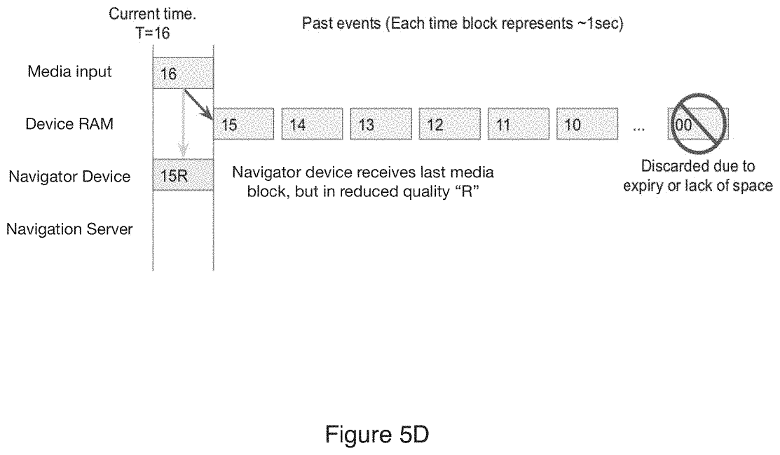

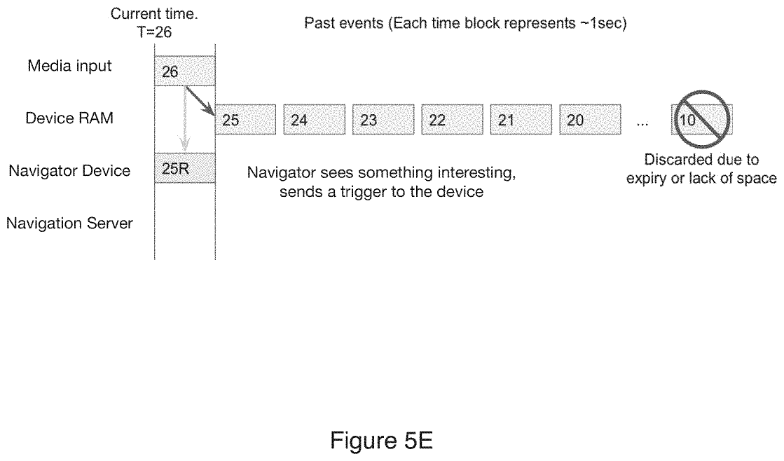

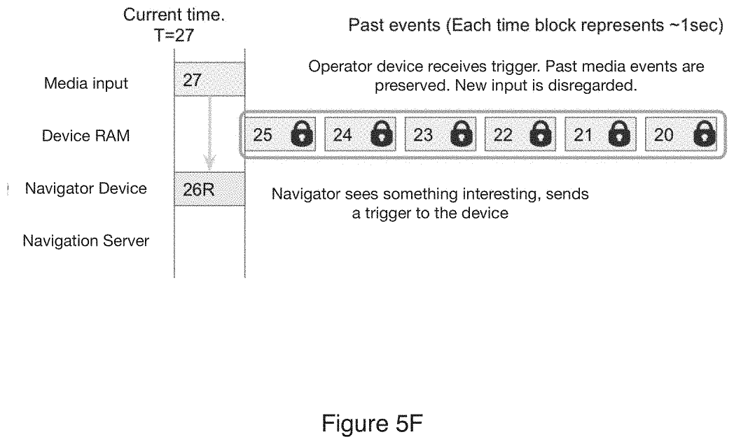

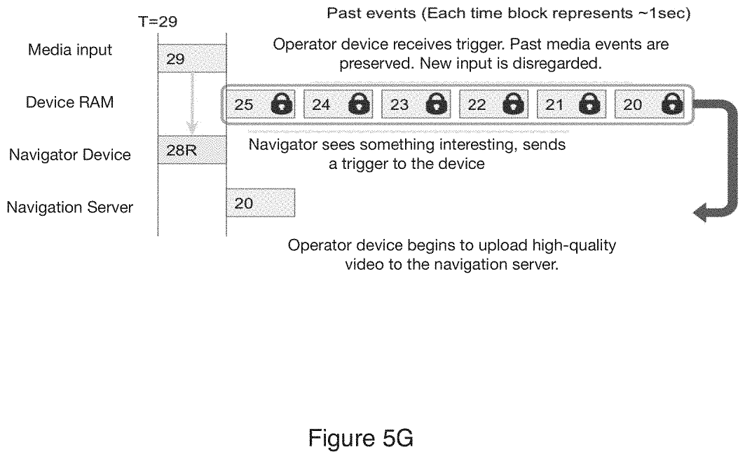

FIGS. 5A, 5B, 5C, 5D, 5E, and 5F are diagrams illustrating one example embodiment of a time-based flow of the navigation process, in accordance with some of the systems and methods herein. The current time T is represented in the diagram. The “present” is represented by events occurring between the two vertical bars on the left side. Various blocks of information are moved around the different locations (or “stations”) listed on the left (i.e., media input, device RAM, navigator device, and navigation server).

At FIG. 5A, current time is T=0. Media block 00 is introduced as information input into the operator device.

At FIG. 5B, current time is T=01. The navigator receives the last media block 00, but in reduced quality “R”. Thus, navigator has 00R in memory. Media input has the incoming media block 01.

At FIG. 5C, the current time is T=06. Media input has the incoming media block 06. The navigator receives the last media block 05, but in reduced quality “R” as 05R. The device RAM holds blocks 00-05.

At FIG. 5D, the current time is T=16. The media input has the incoming media block 16. The device ram holds media blocks 10-15. Media blocks 00-09 have been discarded due to expiry or lack of space. The navigator receives the last media block 15, but in reduce quality “R” as 15R.

At FIG. 5E, the current time is T=26. The media input has media block 26. The operator device RAM holds media blocks 20-25, with media blocks 10-24 being discarded due to expiry or lack of space. The navigator sees something interesting at media block 25R, and sends a trigger to the device.

At FIG. 5F, the current time is T=27. The media input has incoming media block 27. The operator device receives the trigger, and past media events in the form of media blocks 20-25 are preserved. New input is disregarded. The navigator has media block 26R but disregards it.

Finally, at FIG. 5G, the current time is T=29. The media input has incoming media block 29. Since the operator device received the trigger, past media events in the form of media blocks 20-25 are preserved. The operator device begins to upload high-quality video blocks 20-25 to the navigation server.

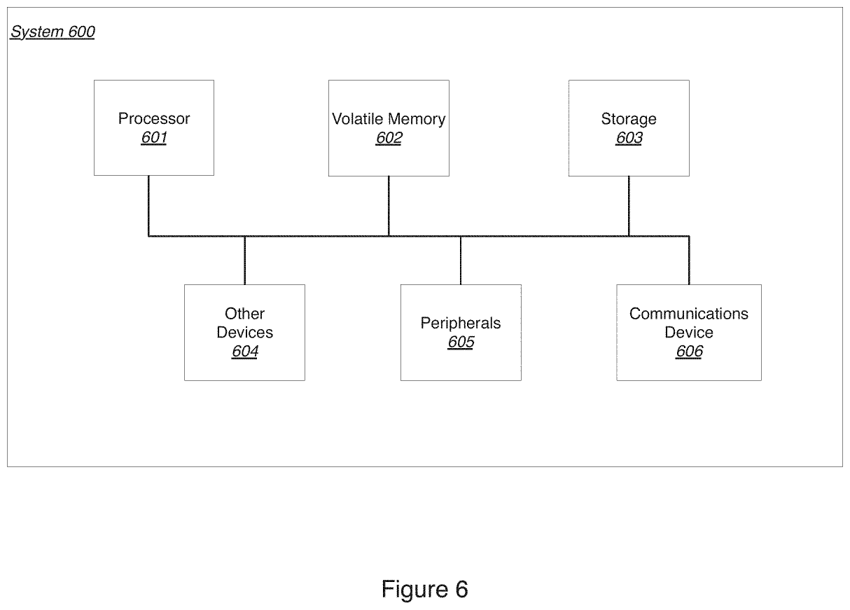

FIG. 6 is a diagram illustrating an exemplary computer that may perform processing in some embodiments. Exemplary computer 600 may perform operations consistent with some embodiments. The architecture of computer 600 is exemplary. Computers can be implemented in a variety of other ways. A wide variety of computers can be used in accordance with the embodiments herein.

Processor 601 may perform computing functions such as running computer programs. The volatile memory 602 may provide temporary storage of data for the processor 601. RAM is one kind of volatile memory. Volatile memory typically requires power to maintain its stored information. Storage 603 provides computer storage for data, instructions, and/or arbitrary information. Non-volatile memory, which can preserve data even when not powered and including disks and flash memory, is an example of storage. Storage 603 may be organized as a file system, database, or in other ways. Data, instructions, and information may be loaded from storage 603 into volatile memory 602 for processing by the processor 601.

The computer 600 may include peripherals 605. Peripherals 605 may include input peripherals such as a keyboard, mouse, trackball, video camera, microphone, and other input devices. Peripherals 605 may also include output devices such as a display. Peripherals 605 may include removable media devices such as CD-R and DVD-R recorders/players. Communications device 606 may connect the computer 100 to an external medium. For example, communications device 606 may take the form of a network adapter that provides communications to a network. A computer 600 may also include a variety of other devices 604. The various components of the computer 600 may be connected by a connection medium 610 such as a bus, crossbar, or network.

In some embodiments, an intermediary component may be introduced between the operator device and the server, such as, e.g., a docking station or charging cradle. In some embodiments, the intermediary component can contain parts of the computer system. In some embodiments, while the intermediary component is physically detached from the operator device, it can function in parallel, in conjunction, or in addition to the operator device.

While the invention has been particularly shown and described with reference to specific embodiments thereof, it should be understood that changes in the form and details of the disclosed embodiments may be made without departing from the scope of the invention. Although various advantages, aspects, and objects of the present invention have been discussed herein with reference to various embodiments, it will be understood that the scope of the invention should not be limited by reference to such advantages, aspects, and objects. Rather, the scope of the invention should be determined with reference to patent claims.

What is claimed:

1. A method for remotely triggering the capture of media data, the method comprising:
Connecting a navigator device at a server with an operator device over a network, wherein the operator device is handled by an operator, and wherein the operator device comprises a speaker and microphone configured to facilitate two-way communications with the navigator device over the network;
receiving, at the server, streaming media data from the operator device; and
concurrent to receiving the streaming media data:
analyzing the streaming media data to determine whether a match for one or more predefined visual landmarks can be identified within the streaming media data,
upon a determination that a match for the one or more predefined visual landmarks cannot be identified within the streaming media data, communicating, via the navigator device, instructions for the operator to reposition the operator device, and
upon a determination that a match for the one or more predefined visual landmarks can be identified within the streaming media data, triggering capture of the streaming media data with the one or more predefined visual landmarks.

2. The method of claim 1, further comprising:
concurrent to receiving the streaming media data:
analyzing the streaming media data to determine whether a predefined threshold for media quality is met or exceeded.

3. The method of claim 2, further comprising:
concurrent to receiving the streaming media data:
upon a determination that the predefined threshold for media quality is neither met nor exceeded, sending one or more instructions to the operator device for the operator to make one or more adjustments and send new streaming media data.

4. The method of claim 1, further comprising:
concurrent to receiving the streaming media data:
automatically processing the streaming media data on the server to improve media quality.

5. The method of claim 1, further comprising:
receiving, at the server, one or more voice statements from the operator of the operator device via the microphone of the operator device; and
in response to receiving the one or more voice statements, sending one or more instructions to the operator device based on the one or more voice statements and the streaming media data.

6. The method of claim 1, wherein the operator device is configured to capture media data in high resolution and generate low resolution versions of the streaming media data, and wherein the received streaming media data comprises the low resolution versions of the streaming media data.

7. The method of claim 6, wherein triggering capture of the streaming media data with the one or more predefined visual landmarks comprises sending high resolution versions of the streaming media data with the one or more predefined visual landmarks to the server for storage.

8. The method of claim 1, wherein communicating instructions for the operator to reposition the operator device comprises sending voice commands from the navigator device to the operator device for playback on the speaker of the operator device.

9. The method of claim 1, wherein the operator handling the operator device comprises the operator grasping and manipulating the operator device in relation to a media target.

10. The method of claim 1, wherein the media target comprises an eye.

11. The method of claim 1, wherein the operator device comprises a proximity sensor configured to measure a distance between the operator device and the media target.

12. The method of claim 11, wherein the operator device is configured to provide feedback to the operator if the distance between the operator device and the media target does not fall within a predetermined range of distance.

13. The method of claim 11, further comprising:
receiving, at the server via the operator device, the distance between the operator device and the media target;
determining whether the distance between the operator device and the media target falls within a predetermined range of distance; and
upon determining that the distance between the operator device and the media target does not fall within the predetermined range of distance, sending one or more instructions for the operator to reposition the operator device.

14. The method of claim 1, wherein the operator device comprises a sensor configured to provide multi-dimensional orientation information to the server.

15. The method of claim 14, further comprising:
receiving, at the server, the multi-dimensional orientation information; and
generating, based on the multi-dimensional orientation, one or more multi-dimensional surface topology maps, one or more stereoscopic images, or a combination thereof.

16. A non-transitory computer-readable medium containing instructions for remotely triggering the capture of media data, the instructions for execution by a computer system, the non-transitory computer-readable medium comprising:
instructions for connecting a navigator device at a server to an operator device over a network, wherein the operator device is handled by an operator, and wherein the operator device comprises a speaker and microphone configured to facilitate two-way communications with the navigator device over the network;
instructions for receiving, at the server, streaming media data from the operator device; and
concurrent to receiving the streaming media data:
instructions for analyzing the streaming media data to determine whether a match for one or more predefined visual landmarks can be identified within the streaming media data,
upon a determination that a match for the one or more predefined visual landmarks cannot be identified within the streaming media data, instructions for communicating, via the navigator device, instructions for the operator to reposition the operator device, and
upon a determination that a match for the one or more predefined visual landmarks can be identified within the streaming media data, instructions for triggering capture of the streaming media data with the one or more predefined visual landmarks.

17. The non-transitory computer-readable medium of claim 16, further comprising:
concurrent to receiving the streaming media data:
instructions for analyzing the streaming media data to determine whether a predefined threshold for media quality is met or exceeded.

18. The non-transitory computer-readable medium of claim 17, further comprising:
concurrent to receiving the streaming media data:
upon a determination that the predefined threshold for media quality is neither met nor exceeded, instructions for sending one or more instructions to the operator device for the operator to resend the streaming media data at a higher media quality.

19. The non-transitory computer-readable medium of claim 16, further comprising:
concurrent to receiving the streaming media data:
instructions for automatically processing the streaming media data on the server to improve media quality.

20. The non-transitory computer-readable medium of claim 16, further comprising:
instructions for receiving, at the server, one or more voice statements from the operator of the operator device via the microphone of the operator device; and
in response to receiving the one or more voice statements, instructions for sending one or more instructions to the operator device based on the one or more voice statements and the streaming media data.
